(19) United States
(12) Patent Application Publication
(10) Pub. No.: US 2017/0014599 A1
(43) Pub. Date: Jan. 19, 2017

(54) DEVICE FOR DISPENSING A FLEXIBLE MEMBER

(71) Applicant: SMITHS MEDICAL ASD, INC., Plymouth, MN (US)

(72) Inventors: Andrew Crisman, Shoreview, MN (US); Alysa Lauren Granata, Minneapolis, MN (US)

(73) Assignee: SMITHS MEDICAL ASD, INC., Plymouth, MN (US)

(21) Appl. No.: 15/189,196

(22) Filed: Jun. 22, 2016

Related U.S. Application Data
(60) Provisional application No. 62/192,238, filed on Jul. 14, 2015.

(51) Int. Cl. A61M 25/01; A61M 39/08; A61M 39/10; A61M 25/09

(52) U.S. Cl. CPC A61M 25/0113; A61M 25/09041; A61M 39/08; A61M 39/10; A61M 2039/087

(57) ABSTRACT

An introducer for dispensing a flexible member such as a catheter has a receptacle extension having a plurality of sections. Some of the sections are configured to have their own given dimensions. The dimensions may be in the form of a given tapered conical configuration for each of the sections so that those sections each are adapted to only fittingly mate with the receptacle of a counterpart connector that has a configuration complementary to the given tapered conical configuration. The introducer is thus usable with connectors of different configurations.

### FIELD OF THE INVENTION

The present invention relates to dispensing devices for introducing longitudinal flexible members such as catheters and guidewires into subject patients, and in particular relates to an introducer that has a dispenser or receptacle end adapted to fittingly mate with different types of connectors.

### BACKGROUND OF THE INVENTION

To introduce a longitudinal flexible member such as a catheter into a subject patient, oftentimes a cannula is inserted into the patient with the tip or the patient end of the cannula being maneuvered to a desired location inside the patient. The user end of the cannula is usually attached or integrated to one end of a hub which other end is a connector. Conventionally, the hub connector is a luer connector designed in accordance with ISO (International Standard Organization) Standard 594-2. The cannula is connected to an introducer that has a counterpart luer connector, so that the luer connector at the hub of the cannula is coupled to the luer connector of the introducer. Once the introducer is connected to the cannula, a catheter, guidewire or other types of tubings may be inserted into the introducer and from there be guided by the cannula to the desired location within the patient.

The catheter may be used to provide medication to the patient, or to withdraw fluid from the patient. So, too, a guidewire may be introduced into the patient by means of the introducer so that, once in place, the cannula, as well as the introducer, are removed from the patient leaving the guidewire in place. Thereafter, the guidewire is used to guide a device, such as for example an epidural needle or another tubing into the patient. Thus, prior to the instant invention, an introducer for dispensing a guidewire or catheter has a luer connector that is connectable to a counterpart luer connector of a patient interface device such as for example a cannula for guiding a catheter or guidewire to the patient. The connector for the introducer may be a male luer and its counterpart connector at the patient cannula may be a female luer, or vice versa. One such introducer is being sold by the assignee of the instant invention under the trade name Logicath Introducer.

There are instances where connectors having configurations different from the male/female configurations of the luer connectors are used in a medical procedure. For example, if it is desirable to use a device having a connector other than a luer connector to introduce an echogenic catheter or a particular type of guidewire and/or flexible tubing into a patient, an introducer with a counterpart non-luer connector would have to be used. Thus, both luer and non-luer introducers are required when devices with luer connectors and non-luer connectors are required in a medical procedure. The use of connectors that have a configuration different from the configuration of the conventional luer connectors is being contemplated by the medical industry under proposed International Standard ISO 80369.

### SUMMARY OF THE PRESENT INVENTION

The instant invention eliminates the need for using different introducers when devices with different types of connectors are used.

The inventive dispenser introducer has a receptacle end having an elongated extension that has a plurality of sections therealong including at least two adjacent sections that have different tapered conical dimensions. The elongated extension may also be referred to as a receptacle extension, a dispenser extension or simply an extension hereinafter. One of the sections is formed to have a tapered conical connector with a dimension adapted to fittingly mate with the receptacle of a conventional luer connector. Thus, the tapered conical configuration of the one section is dimensioned such that it is a counterpart of, or complementary to, the tapered conical receptacle of a luer connector. The other of the sections is dimensioned to have a tapered conical configuration that does not fittingly mate with a luer receptacle but is fittingly matable with a connector that has a receptacle having a tapered conical configuration complementary to that of the other section. Since the tapered conical configuration of the non-luer connector section has a dimension that is smaller than the section with the tapered conical configuration for mating with a luer connector, the non-luer section is positioned closer to the distal end of the extension of the inventive introducer. The section that has the tapered conical configuration adapted to mate with a luer connector is formed rearwardly or proximally along the extension so as to be distant from the distal end of the extension. Thus constructed, the sections of the extension that are correspondingly distant from and closer to the distal end of the extension are adapted to accept a luer connecter and a non-luer connector, respectively.

The inventive introducer also includes a guide at its proximal portion that has a longitudinal aperture. The guide is separated from the extension of the introducer by a base of the body that is in the form of a platform having a raised portion. The aperture at the guide and the through bore at the extension are in longitudinal alignment so that a longitudinal flexible member such as for example a catheter may be threaded through the aperture of the guide and the through bore of the extension. As the portion of the catheter superposed over the platform has a portion thereof in contact with the raised portion, a user can press the catheter against the raised portion and use the raised portion as a contact point to feed the catheter through the bore of the extension into the device coupled thereto. The user can also move the catheter bidirectionally or in a bidirectional manner by maneuvering the catheter against the raised portion.

To enable a user to hold the introducer, a handle extends integrally from the bottom of the base, and is positioned such that the user can hold the introducer with her hand while using her thumb to maneuver the movement of the catheter by pressing the catheter against the raised portion at the base platform.

The present invention is therefore directed to an apparatus for introducing a longitudinal flexible member to a device adapted to convey the member, comprising:
an extension adapted to mate with a plurality of connectors having different configurations, the extension having a distal end and a through bore, at least two sections formed along the extension, each of the sections formed with a given tapered conical configuration so as to be matable only with a corresponding connector having a tapered conical configuration complementary to the given tapered conical configuration, the tapered conical configuration of the section close to the distal end of the extension having a smaller dimension than the tapered conical configuration of the section distant from the distal end of the extension;
a body having a distal portion whereat the extension integrally extends and a proximal portion having an integral guide, the guide having an aperture in longitudinal alignment with the bore at the extension, the body including a base sandwiched by the extension and the guide whereon the flexible member is maneuverable to move bi-directionally between the extension and the guide;
wherein the flexible member is threaded through the aperture of the guide and the bore of the extension when being introduced to the device; and
wherein when the extension is coupled to a connector having a tapered conical configuration complementary to one of the sections, the connector is fittingly mated to said one section so that the flexible member may be introduced to the device for conveyance.

The instant invention is further directed to an apparatus for introducing a longitudinal flexible member to a device, comprising:
a one piece body having an extension and a guide integrally connected by an elongate base, the extension having a distal end and a through bore, at least one and other sections formed along the extension, the one section being closer to the distal end of the extension and has one given tapered conical configuration, the other section being distant to the distal end of the extension and has an other given tapered conical configuration, the one and other sections adapted to fittingly mate with corresponding connectors having tapered conical configurations complementary to the one and other given tapered conical configurations, respectively, the guide having an aperture in longitudinal alignment with the bore at the extension so that the flexible member is threadable through the aperture of the guide and the bore of the extension when being introduced to the device, the flexible member maneuverable to move bi-directionally along the base between the extension and the guide, the extension fittingly matable to a connector having a tapered conical configuration complementary to the tapered conical configuration of either of the one and other sections.

The instant invention is furthermore directed to a method of introducing a longitudinal flexible member to a passage of a device using an introducer having a one piece body including an extension and a guide integrally connected by an elongate base, the extension having a distal end, a through bore and at least one and other sections formed therealong, the one section having one given tapered conical configuration and the other section having an other given tapered conical configuration, the one and other sections adapted to fittingly mate with corresponding connectors having tapered conical configurations complementary to the one and other given tapered conical configurations, respectively, the guide having an aperture in longitudinal alignment with the bore at the extension, the method comprising the steps of:
(a) connecting the device to the introducer by coupling a connector of the device to the extension;
(b) ensuring the connector of the device has a tapered conical configuration complementary to the one or other section by friction fitting the connector to the one or other section;
(c) threading a distal end of the flexible member through the aperture of the guide and the bore of the extension;
(d) maneuvering the flexible member along the base to introduce the flexible member into the passage of the device; and
(e) continue to feed the flexible member along the device until the distal end of the flexible member extends beyond the end of the passage of the device.

### BRIEF DESCRIPTION OF THE FIGURES

The present invention will become apparent and the invention itself will be best understood with reference to the following description of the invention taken in conjunction with the accompanying drawings, wherein:

FIG. 1 is a perspective view of the inventive introducer;

FIG. 2 is an enlarged perspective view of the receptacle extension of the inventive introducer;

FIG. 3 is a top cross-sectional view of the receptacle extension of the inventive introducer having fittingly mated to one of its sections the connector at the hub of a device adapted to interface with a subject patient;

FIG. 4 is a perspective view of the receptacle extension of the inventive introducer showing the section to which the connector of the device shown in FIG. 3 is friction fitted;

FIG. 5 is a top cross-sectional view of the receptacle extension of the inventive introducer having another connector fittingly mated to another section therealong; and

FIG. 6 is a perspective view of the receptacle extension of the introducer of the instant invention showing in particular the section to which the connector of the device shown in FIG. 5 is friction fitted.

### DETAILED DESCRIPTION OF THE INVENTION

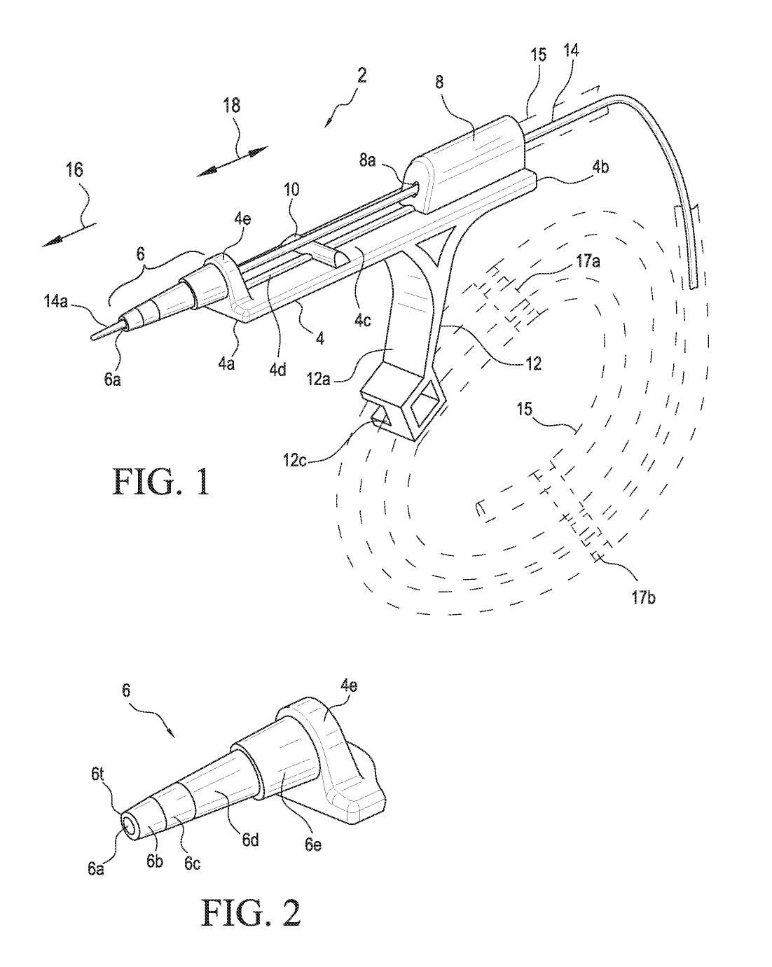

With reference to FIG. 1, the inventive introducer 2 is shown to have a body 4 that has at its distal portion 4a a substantially tapered cone shape extender or receptacle extension 6. The body also has a proximal portion 4b where there is a longitudinal guiding extension or simply guide 8. The portion of the body 4 that connects, or is sandwiched by, extension 6 and guide 8, except for a raised portion 10, has a substantially flat shape in the form of a platform 4c. A longitudinal groove 4d runs along platform 4c, except where it intersects raised portion 10. Groove 4d is dimensioned to accept and provide a guide path to a longitudinal flexible member, such as for example a catheter, that is being moved along the platform 4c. Extending from the bottom of body 4 is a handle 12 that has a curvilinear portion 12a that enables a user to grasp handle 12 by means of her fingers against her palm while freeing her thumb to maneuver the exemplar catheter, a guidewire or a tubing, along the platform 4c, as will be discussed further below. It should be appreciated that introducer 2 as shown in FIG. 1 may be a one piece unitary molded device that may be made from polyurethane, polypropylene or some other plastics material adaptable for medical uses.

Further with reference to FIG. 1, it can be seen that guide 8 has an aperture 8a at its distal end through which a longitudinal member, for example a catheter 14, is threaded. Although not shown, at the proximal end of guide 8 is an opening that forms a channel having a diameter larger than that of aperture 8a that extends part way into guide 8. The channel may be used for fittingly accept a distal portion of a guide tube 15, shown in dotted lines, that houses catheter 14 to protect it from the environment and also prevents possible kinking and tangling of the catheter. Only a portion of catheter 14 is shown. Guide tube 15 may be rolled into a coil and may be held by a number of holders such as 17a and 17b. A section of guide tube 15 is snap fitted to a side grasp opening 12c at handle 12 so that guide tube 15, and therefore catheter 14 housed therein, is held in position relative to introducer 2. It should be noted that tube 15 may also be used to house a guidewire or a tubing for use with introducer 2.

As illustrated in FIG. 1, catheter 14 passes through aperture 8a of guide 8, extends along base platform 4c and passes into and extends out of a through bore 6a in extension 6, as per shown by its distal end 14a. In use, a user grasps handle 12 with her hand while uses her thumb to press catheter 14 against raised portion 10 to feed the catheter 14 in the forward or distal direction as indicated by directional arrow 16. Extension 6 may be coupled to a patient interface device, for example a cannula that has its patient end positioned in a desired location in a subject patient, so that the catheter may be conveyed to the desired position inside the patient. In addition to being able to move the catheter in the forward direction, the user can also move catheter 14 bidirectionally by using the raised portion 10 as a contact point to movably maneuver catheter 14 toward either extension 6 or guide 8, as per shown by bidirectional arrow 18.

FIG. 2 is an enlarged view of extension 6. As shown, exemplar extension 6 has a distal end 6t and four successively adjacent sections 6b, 6c, 6d and 6e extending proximally away from distal end 6t. Section 6b begins at distal end 6t and has a blunt semi-rounded conical shape such that it can readily enter into a receptacle of a connector. Section 6c extends proximally from sections 6b and may have a taper. The diameter along section 6c may or may not increase in size relative to the diameter at the proximal end of section 6b. Section 6d, on the other hand, is tapered and dimensioned to conform with a reverse taper of a particular type of connector, and therefore has a given or predetermined tapered conical configuration. Although shown as different sections, in practice, sections 6c and 6d may be consolidated into a single continuous tapered cylindrical section.

Section 6e integrally extends proximally from its adjacent section 6d and is therefore further away, or distant, from distal end 6t of extender 6 than section 6d. Section 6e is also taperedly formed to have its own given tapered conical configuration and is integrally connected to an upright portion 4e of base 4. For exemplar extension 6, section 6e has a cross-sectional dimension that is greater than the cross sectional dimension of section 6d, and in particular is configured to have a tapered conical configuration that allows it to fittingly mate with a counterpart receptacle of a conventional luer connector. Putting it differently, section 6e is configured to friction fit with a counterpart conventional luer connector. It should be appreciated that the respective cross-sectional dimensions of sections 6d and 6e progressively change along their lengths, for example getting progressively larger toward the proximal end of extension 6 as shown in FIG. 2. For exemplar extension 6, section 6e is a male connector while the connector to which it mates is a female luer connector. The dimensions of the receptacle of a conventional luer connector are well known and are specified in ISO Standard 594-2.

On the other hand, the tapered conical configuration of section 6d is designed to have a non-luer configuration so that it is not fittingly matable with a counterpart luer connector. Instead, section 6d is designed to have a dimension that allows it to only fittingly mate with, or friction fit to, a connector that has a complementary tapered conical configuration. Thus, section 6d is not fittingly matable with a conventional female luer connector but is matable with a counterpart connector that has a receptacle having a configuration that is complementary to the non-luer configuration of section 6d. An exemplar use of such non-luer connector may be for an epidural cannula that has a connector specifically designed not to be connectable to a counterpart luer connector.

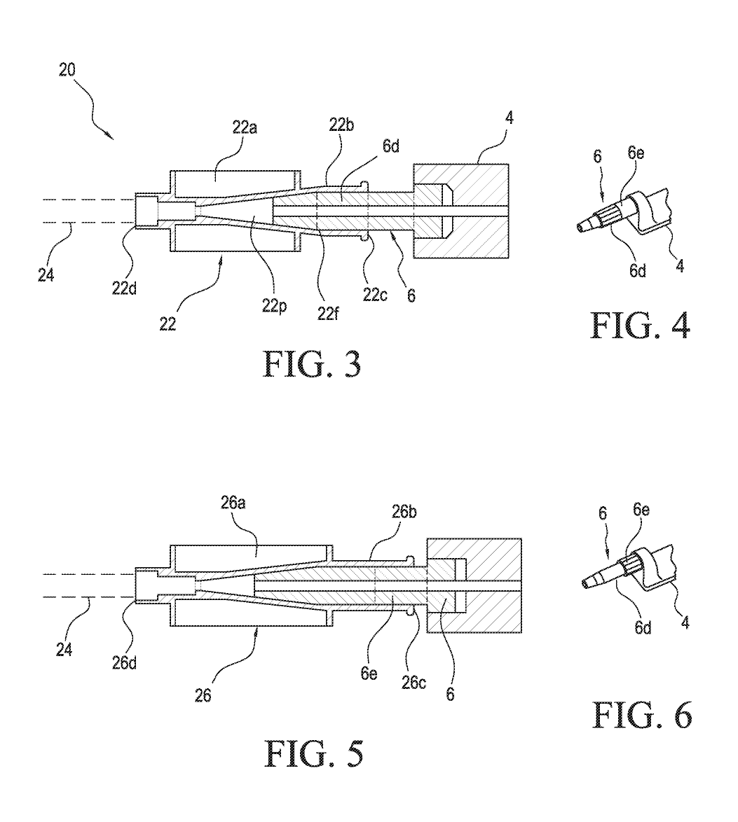

FIG. 3 is a top cross-sectional view showing the coupling of extension 6 of the inventive introducer with a device 20 that has a specially designed connector 22. As shown, device 20 presumably is used to interface with a subject patient and has a cannula 24 (shown in dotted lines) connected to the patient end of a hub 22a and a connector 22b at the non-patient end. Hub 22a has a passage 22p that connects connector end 22c to the cannula end 22d. Passage 22p is tapered from within hub 22a to its connector end 22c and is dimensioned to have a tapered conical configuration that is complementary to the tapered conical configuration of section 6d of extension 6d of introducer 6. For illustration purposes, section 6d is shown in dotted lines in FIG. 3 and is shown lengthwise lined in FIG. 4. Given the complementary tapered conical configurations of section 6d of extension 6 and the receptacle of connector 22b of hub 22 of device 20, extension 6 is fittingly mated with, or friction fitted to, connector 22. Thus, a through passage is established from the through bore of extension 6 through hub 22a into cannula 24. As a result, catheter 14 shown in FIG. 1 can be maneuvered by a user along platform 4c into cannula 24 and be guided by the cannula, with the patient end of the catheter exiting from the patient end of the cannula, to the desired location in the subject patient.

FIG. 5 is an illustration of the coupling of extension 6 to a conventional luer connector 26. As shown, connector 26 has a hub 26a that has a connector 26d with a connector end 26c. A cannula 24 (in dotted lines) is attached a cannula end 26d of hub 26a. Also shown in the top cross-sectional view of FIG. 5 is a taper from within hub 26a that increases towards connector portion 26b. The receptacle opening of the luer connector 26b has a dimension that is greater than the receptacle opening of the connector portion 22b of connector 22 shown in FIG. 3. Accordingly, connector portion 26b is not fittingly matable with section 6d of extension 6 when connector 26 and extension 6 are moved relatively toward each other. As the dimension of the tapered conical configuration of section 6e is greater than the dimension of the tapered conical configuration of section 6d, the connector portion 26b, which has a tapered conical configuration complementary to that of section 6e, would bypass section 6d to fittingly mate with section 6e, as per shown by the dotted lines in FIG. 5 and the lined section 6e shown in FIG. 6. Thus, the inventive introducer is adapted to accept a plurality of connectors that have different configurations. This is advantageous as there is no need to have separate introducers for use with different types of connectors that may be required during a medical procedure.

In addition to being able to be used with a catheter, the introducer of the instant invention is adapted to be used with a guidewire or other tubings. For example, in the case of a guidewire, after the connector of a cannula device has been fittingly mated to one of the sections of extension 6, the guidewire is fed into the connector so as to be conveyed to the desired location within the subject patient. Thereafter, the introducer and the cannula device are removed. With both the introducer and the patient interface device removed, the guidewire may then be used to guide another device, such as an echogenic catheter or an epidural/neural needle along the guidewire into the patient. After the device is correctly positioned within the subject patient, the guidewire is removed so that the catheter or needle remains in place in the subject patient.

The present invention is subject to many variations, modifications and changes in details. For example, the catheter selected for use may have a particular type of connector for coupling to the extension of the introducer. Further, instead of only two sections of differently dimensioned configurations, additional section(s) with its own given tapered conical configuration may be added to the extension to provide additional flexibility to the inventive introducer, provided that the extension has sufficient length and the different connectors are adapted to be fitted to separate locations along the extension. The number of sections to be added to the extension of the introducer may be dependent on the number of different connectors and the respective configurations of those connectors.

1. Apparatus for introducing a longitudinal flexible member to a device adapted to convey the member, comprising:
an extension adapted to mate with a plurality of connectors having different configurations, the extension having a distal end and a through bore, at least two sections formed along the extension, each of the sections formed with a given tapered conical configuration so as to be matable only with a corresponding connector having a tapered conical configuration complementary to the given tapered conical configuration, the tapered conical configuration of the section close to the distal end of the extension having a smaller dimension than the tapered conical configuration of the section distant from the distal end of the extension;
a body having a distal portion whereat the extension integrally extends and a proximal portion having an integral guide, the guide having an aperture in longitudinal alignment with the bore at the extension, the body including a base sandwiched by the extension and the guide whereon the flexible member is maneuverable to move bi-directionally between the extension and the guide;
wherein the flexible member is threaded through the aperture of the guide and the bore of the extension when being introduced to the device; and
wherein when the extension is coupled to a connector having a tapered conical configuration complementary to one of the sections, the connector is fittingly mated to said one section so that the flexible member may be introduced to the device for conveyance.

2. Apparatus of claim 1, wherein the two sections are adjacent to each other, the section distant from the distal end of the extension having a tapered conical configuration adapted to fittingly mate with a luer connector.

3. Apparatus of claim 1, wherein the section close to the distal end of the extension has a tapered conical configuration not fittingly matable with a luer connector.

4. Apparatus of claim 1, wherein the body has a handle extending from below the base to enable a user to hold the apparatus.

5. Apparatus of claim 1, wherein the base comprises a raised portion onto which a user can press the flexible member against to move the member bi-directionally along the base.

6. Apparatus of claim 1, wherein the flexible member comprises a catheter, a guidewire or a tubing.

7. Apparatus of claim 1, wherein the device comprises a cannula having a hub connector, the cannula having a distal end positioned at a desired location in the subject, the longitudinal flexible member passing into and guided by the cannula to the desired location.

8. Apparatus for introducing a longitudinal flexible member to a device, comprising:
a one piece body having an extension and a guide integrally connected by an elongate base, the extension having a distal end and a through bore, at least one and other sections formed along the extension, the one section being closer to the distal end of the extension and has one given tapered conical configuration, the other section being distant to the distal end of the extension and has an other given tapered conical configuration, the one and other sections adapted to fittingly mate with corresponding connectors having tapered conical configurations complementary to the one and other given tapered conical configurations, respectively, the guide having an aperture in longitudinal alignment with the bore at the extension so that the flexible member is threadable through the aperture of the guide and the bore of the extension when being introduced to the device, the flexible member maneuverable to move bi-directionally along the base between the extension and the guide, the extension fittingly matable to a connector having a tapered conical configuration complementary to the tapered conical configuration of either of the one and other sections.

9. Apparatus of claim 8, wherein the other section has a tapered conical configuration adapted to fittingly matable with a luer connector.

10. Apparatus of claim 8, wherein the one section has a tapered conical configuration not fittingly matable with a luer connector.

11. Apparatus of claim 8, wherein the body has a handle to enable a user to hold the apparatus.

12. Apparatus of claim 8, wherein the base comprises a raised portion onto which a user can press the flexible member against to move the member bi-directionally along the base.

13. Apparatus of claim 8, wherein the flexible member comprises a catheter, a guidewire or a tubing.

14. Apparatus of claim 8, wherein the device comprises a cannula having a hub connector for mating with the one or other section of the extension.

15. A method of introducing a longitudinal flexible member to a passage of a device using an introducer having a one piece body including an extension and a guide integrally connected by an elongate base, the extension having a distal end, a through bore and at least one and other sections formed therealong, the one section having one given tapered conical configuration and the other section having an other given tapered conical configuration, the one and other sections adapted to fittingly mate with corresponding connectors having tapered conical configurations complementary to the one and other given tapered conical configurations, respectively, the guide having an aperture in longitudinal alignment with the bore at the extension, the method comprising the steps of:
(a) connecting the device to the introducer by coupling a connector of the device to the extension;
(b) ensuring the connector of the device has a tapered conical configuration complementary to the one or other section by friction fitting the connector to the one or other section;
(c) threading a distal end of the flexible member through the aperture of the guide and the bore of the extension;
(d) maneuvering the flexible member along the base to introduce the flexible member into the passage of the device; and
(e) continue to feed the flexible member along the device until the distal end of the flexible member extends beyond the end of the passage of the device.

16. Method of claim 15, wherein step (d) comprises the step of;
pressing the flexible member against a raised portion along the base to move the longitudinal flexible member bi-directionally along the base.

17. Method of claim 15, further comprising the step of:
selecting the flexible member to be used depending on whether the one of other sections is fittingly mated by the connector.

18. Method of claim 17, further comprising the step of:
selecting the longitudinal flexible member to be used from among a catheter, a guidewire and a tubing.
